(19) United States
(12) Patent Application Publication
(10) Pub. No.: US 2025/0248957 A1
(43) Pub. Date: Aug. 7, 2025

(54) CELL DIFFERENTIATION

(71) Applicant: JOHN HEMMING TRADING LTD, Birmingham (GB)

(72) Inventor: John HEMMING, Birmingham (GB)

(21) Appl. No.: 18/856,013

(22) PCT Filed: Mar. 31, 2023
(86) PCT No.: PCT/GB2023/050859

§ 371 Date: Oct. 10, 2024

(30) Foreign Application Priority Data
Apr. 14, 2022 (GB) 2205525.5
Jun. 20, 2022 (GB) 2209046.8
Dec. 23, 2022 (GB) 2219693.5

(51) Int. Cl. A61K 31/194; A61K 31/11; A61K 31/197; A61K 31/385; A61K 31/4045; A61K 31/439; A61K 31/506; A61K 31/522; A61P 17/14

(52) U.S. Cl. CPC A61K 31/194; A61K 31/11; A61K 31/197; A61K 31/385; A61K 31/4045; A61K 31/439; A61K 31/506; A61K 31/522; A61P 17/14

(57) ABSTRACT

The invention relates to methods for promoting and maintaining cell differentiation and gene expression in a subject, and ensuring that longer genes are able to function, the method comprising administering citrate to a subject. The invention also relates to compositions for use in said methods.

### TECHNICAL FIELD OF THE INVENTION

The invention relates to methods for promoting and maintaining cell differentiation and gene expression in a subject, and ensuring that longer genes are able to function, the method comprising administering citrate to a subject. The invention also relates to compositions for use in said methods.

### BACKGROUND OF THE INVENTION

Cell differentiation and maintenance of differentiated cells is vital in multicellular organisms and underpins a variety of biological processes. Cell differentiation and maintenance of differentiated cells relies on the ability of cells to promote and maintain gene expression.

Differentiation of stem cells is highly important for normal biological functioning. Stem cells are undifferentiated cells which have the ability to renew and to differentiate into a variety of specialised cell types. Stem cells are often quiescent, meaning they exist in a reversible state of cell cycle arrest. Importantly, stem cells are able to exit quiescence in response to particular stimuli allowing them to rapidly expand and differentiate to produce a variety of specialised cell types. When stem cells fail to differentiate in response to appropriate stimuli, they become arrested in a quiescent state. Stem cells play a role in various diseases, and failure of stem cells to differentiate can have serious impacts on health. Failure of stem cells to differentiate may contribute to various inflammatory diseases such as atherosclerosis, and musculoskeletal diseases such as sarcopenia and osteoporosis. Sarcopenia is associated with a progressive reduction in the regenerative capacity of skeletal muscle stem cells. Osteoporosis is a condition that leads to weakened bones, typically in older people, and occurs when mesenchymal stem cells exhibit reduced ability to differentiate into osteoblasts (which regenerate bone) thereby disrupting the balance between osteoblasts and osteoclasts (which absorb bone) resulting in bone being reabsorbed faster than it is regenerated.

Differentiated cells can undergo dedifferentiation. Dedifferentiation is a process by which differentiated cells become less specialised and revert to a less differentiated stage within the cell lineage. Cell dedifferentiation prevents cells from functioning effectively and can have various therapeutic and cosmetic implications.

Cell differentiation and cell dedifferentiation play important roles in hair growth and loss. Hair follicle stem cells reside in the hair follicle bulge and differentiate to produce the cells required for hair growth during hair growth cycles. Reduced hair growth or increased hair loss may occur when hair follicle stem cells become arrested in a quiescent state. Hair loss may also occur when cells required for hair growth become dedifferentiated, meaning they are no longer able to sustain hair growth.

Gene expression plays a pivotal role in differentiation and underpins the function of differentiated cells. Some cells may be in a differentiated state but fail to express all of the genes required to maintain proper functioning. A shortage of acetyl-CoA in the nucleus may cause a reduction in expression or silencing of genes, e.g. long genes, even in cells which remain in a differentiated state. Reduced gene expression may result in reduced functionality in these cells.

There exists an urgent and unmet need for methods of promoting and maintaining cell differentiation and gene expression. In particular, there is an urgent and unmet need for methods of promoting differentiation of stem cells, particularly stem cells which are arrested in a quiescent state.

### SUMMARY OF THE INVENTION

The Inventor has made the surprising discovery that administering citrate to a subject promotes and maintains cell differentiation and gene expression. Advantageously, the Inventor has found that administration of citrate promotes differentiation of stem cells which have been arrested in a quiescent state. The Inventor has also discovered that administration of citrate helps maintain cells in a differentiated state by reducing the occurrence of cell dedifferentiation, and thereby maintaining cellular function. The Inventor has also discovered that administration of citrate helps to maintain and promote gene expression in cells, e.g. to increase the range of genes expressed by cells.

The invention provides a method of promoting or maintaining gene expression in a subject, the method comprising administering citrate to the subject. Beneficially, citrate administration promotes transcription of genes, particularly genes>10 kb in length which would otherwise be susceptible to stalling and/or premature termination of the RNA Polymerase II complex due to acetyl CoA deficits.

The invention also provides a method of promoting or maintaining cell differentiation and gene expression in a subject, the method comprising administering citrate to the subject.

The invention also provides a method of preventing or reducing an effect of aging in a subject, the method comprising administering citrate to the subject. In some embodiments, preventing or reducing an effect of aging comprises rejuvenating cells. In some embodiments, the method is a cosmetic method of preventing or reducing an effect of aging in a subject. In some embodiments, the effect of aging is one or more of reduced skin integrity (e.g. increased presence or appearance of wrinkles) or hair loss and/or greying. In some embodiments, the effect of aging is atherosclerosis, osteoporosis, sarcopenia or diabetes.

The invention also provides a method of improving the function of cells in a subject, the method comprising administering citrate to the subject. In some embodiments, improving the function of cells comprises rejuvenating cells. Improving the function of cells, or rejuvenating cells, may comprise increasing the expression of long genes (e.g. genes with a length of ≥10 kb). Rejuvenating cells typically means that cells exhibit increased gene expression, particularly of long genes.

In some embodiments, the citrate is administered in combination with an antioxidant. In some embodiments, the antioxidant is selected from melatonin, ethanol, a carotenoid, a flavonoid, ascorbic acid, vitamin E, and grape seed extract.

Typically, administration of citrate in combination with a combination therapy (e.g. an additional agent or red/infrared light) described herein results in a synergistic effect.

In some embodiments, the citrate is administered in combination with an anti-inflammatory agent. In some embodiments, the anti-inflammatory agent is selected from non-steroidal anti-inflammatory drug (e.g. aspirin or ibuprofen), and a corticosteroid.

In some embodiments, the method comprises administering citrate to the subject more than once per day. In some embodiments, the method comprises administering citrate to the subject at least three times per day.

In some embodiments, the citrate is administered as a salt, optionally wherein the salt comprises a cation selected from calcium, magnesium, potassium, and sodium.

In some embodiments, the method further comprises topically administering red or infrared radiation with a wavelength of between 600 and 900 nanometres to the subject. Administration of red or infrared radiation improves mitochondrial efficiency by resonating the copper atoms in Cytochrome C oxidase. Thus, administration of citrate and red or infrared radiation function together to increase acetyl-CoA production. In some embodiments, radiation is administered for up to 20 minutes, up to 15 minutes, up to 10 minutes, or up to 5 minutes. In some embodiments, the radiation is administered between one and four times per day on a particular area of the body, e.g. the scalp.

In some embodiments, the cells are stem cells.

In some embodiments, the cells are hair follicle cells, optionally hair follicle stem cells. In some embodiments, the method comprises promoting hair growth in a subject and/or preventing hair loss in a subject. In some embodiments, the method comprises treating or preventing male pattern baldness. In some embodiments, the method comprises treating or preventing alopecia. In some embodiments, the method comprises treating or preventing greying of hair.

In some embodiments, the method comprises treating or preventing atherosclerosis.

In some embodiments, the method comprises treating or preventing hearing loss.

In some embodiments, the method comprises treating or preventing a condition associated with aging.

In some embodiments, the method comprises treating or preventing osteoporosis.

In some embodiments, the method comprises treating or preventing sarcopenia.

In some embodiments, the method comprises promoting glucose homeostasis in a subject. In some embodiments, the method comprises treating or preventing diabetes.

In some embodiments, the method comprises treating or preventing cancer.

In some embodiments, the method comprises promoting wound healing in a subject.

In some embodiments, the method comprises preventing or treating scar tissue.

In some embodiments, the method comprises improving the condition of the skin. In some embodiments, the method is a cosmetic method for improving the condition of the skin. In some embodiments, improving the condition of the skin comprises reducing the appearance of wrinkles, increasing firmness, and/or reducing the appearance of scars.

In some embodiments, the method comprises improving kidney function. In some embodiments, the method comprises treating or preventing kidney function loss.

In some embodiments, the method comprises improving pancreatic function. In some embodiments, the method comprises treating or preventing pancreatic function loss. In some embodiments, the method comprises improving glucose processing.

In some embodiments, the method comprises administering an anti-cholesterol agent to the subject.

The invention provides a method of promoting histone acetylation in a subject, the method comprising administering citrate to the subject.

In some embodiments, the citrate is administered in combination with: (a) an antioxidant; and/or (b) an anti-inflammatory agent.

In some embodiments: (a) the antioxidant is selected from melatonin, ethanol, a carotenoid, a flavonoid, ascorbic acid, vitamin E, and grape seed extract; and/or (b) the anti-inflammatory agent is selected from non-steroidal anti-inflammatory drug (e.g. aspirin or ibuprofen), and a corticosteroid.

In some embodiments, the method comprises administering multiple doses of citrate to the subject in a 24 hour period.

In some embodiments, citrate is administered at regular intervals throughout one or more 24 hour periods. In some embodiments, citrate is administered at least every 6 hours, at least every 5 hours, at least every 4 hours, at least every 3 hours, at least every 2 hours, or at least every hour during the day.

In some embodiments, the citrate is administered as a salt, optionally wherein the salt comprises a cation selected from calcium, magnesium, potassium, and sodium.

In some embodiments, the method comprises improving cell health in the subject. In some embodiments, improving cell health comprises improving the health of at least one of hair follicle cells, muscle cells, skin cells, and stem cells.

In some embodiments, the method comprises: (i) monitoring inflammation levels in the subject; and (ii) stopping or pausing citrate administration if inflammation levels exceed a threshold level.

In some embodiments, the citrate is administered as a salt, wherein the method comprises: (i) monitoring the level of at least one cation of the citrate salt in a biological sample obtained from the subject; and (ii) stopping or pausing citrate administration if the level of said at least one cation of the citrate salt exceeds a threshold level; optionally wherein the cation is selected from calcium, magnesium, potassium, and sodium.

In some embodiments, the method comprises administering to the subject one or more supplements selected from vitamin D3, boron, zinc, quercetin, β-Nicotinamide mononucleotide (NMN), nicotinamide riboside (NR), sulforaphane, chia seeds, black turkey beans, psyllium husk, hyaluronic acid, berberine, ferulic acid, phosphorylated serine, 25 hydroxyvitamin D (25 (OH) D), aspirin, resveratrol, pterostilbene, pantethine, ketone ester, medium chain triglycerides (MCT), vitamin A, vitamin B, vitamin C, vitamin E or vitamin K (including vitamin K2 MK4, vitamin K2 MK7 and vitamin K2 MK9), fish oil, collagen, curcumin, carnosine, betaine, astaxanthin and grape seed extract.

In some embodiments, the method comprises administering citrate to the subject if the level of C-reactive protein in a biological sample obtained from the subject exceeds a threshold level.

In some embodiments, the citrate is administered systemically.

In some embodiments, the citrate is administered to the subject by sublingual administration, intravenous administration, transdermal administration, or oral administration.

In some embodiments, the anti-inflammatory agent and/or anti-oxidant is administered orally.

In some embodiments, the citrate is formulated for time-release and/or sustained release. In some embodiments, the method comprises administering to the subject one or more histone deacetylase inhibitors, optionally wherein the one or more histone deacetylase inhibitors are selected from Berberine, Curcumin, Quercetin and Pterostilbene. Advantageously, citrate and histone deacetylase inhibitors act synergistically to improve gene expression.

In some embodiments, the method comprises administering to the subject a GSK3B inhibitor, optionally wherein the GSK3B inhibitor is lithium.

In some embodiments, the method comprises administering to the subject acetate or an acetate precursor such as ethanol.

In some embodiments, the method comprises administering to the subject a B vitamin, optionally wherein the B vitamin is selected from vitamin B1, vitamin B2, vitamin B3, vitamin B5, vitamin B6, vitamin B7, vitamin B9 and vitamin B12.

In some embodiments, the method comprises administering to the subject copper. Copper is used as part of the metabolic processes.

In some embodiments, the method comprises administering to the subject pantethine or vitamin B5. Pantethine and vitamin B5 are both Coenxyme A precursors. Pantethine is not rate limited.

In some embodiments, the method comprises administering to the subject caffeine, optionally wherein the caffeine is administered topically. Caffeine is an antiandrogen and enables faster progress with hair regeneration. In some embodiments, administering caffeine reduces or avoids the requirement to increase citrate dosage and/or enables the administration of a lower citrate dosage.

In some embodiments, the method comprises administering to the subject minoxidil. When applied topically, minoxidil increases local blood flow, that both reduces the localised impact of IL-10 and increases the speed of change of cells. In some embodiments, administering minoxidil reduces or avoids the requirement to increase citrate dosage and/or enables the administration of a lower citrate dosage.

In some embodiments, the method comprises administering to the subject vanillin. Vanillin is a metabolite of curcurmin and assists with improving autophagy and mitochondrial quality.

In some embodiments, the method comprises administering to the subject Alpha Lipoic Acid. Alpha Lipoic Acid is a HDAC inhibitor.

In some embodiments, the method comprises administering to the subject Rapamycin. Rapamycin encourages autophagy through inhibiting mTOR and improves mitochondrial quality.

In some embodiments, the method comprises administering to the subject a 3-hydroxy-3-methyl-glutaryl-coenzyme A (HMG) reductase inhibitor. This reduces the use of Acetyl-CoA for cholesterol production thereby leaving more for acetylation.

In some embodiments, the method comprises administering to the subject ribonucleotides. This encourages gene expression without having to increase acetylation excessively.

In some embodiments, the method comprises administering to the subject at least 0.5 g of citrate per day, optionally at least 2 g, at least 3 g, at least 4 g, at least 5 g, at least 10 g, at least 15 g, at least 20 g, at least 30 g, at least 40 g, or at least 50 g of citrate per day. In some embodiments, the method comprises administering at least 2 g of citrate per day, optionally at least 3 g, at least 4 g, at least 5 g, at least 10 g, at least 15 g, at least 20 g, at least 30 g, at least 40 g, or at least 50 g of citrate per day. In some embodiments, the method comprises administering to the subject at least 10 ml of lemon juice per day.

In some embodiments, the citrate is formulated for topical administration. In some embodiments, the citrate is formulated as a liquid. In some embodiments, the liquid comprises a solvent, optionally wherein the solvent is dimethyl sulphoxide (DMSO). The presence of a solvent advantageously encourages absorption of citrate across the cell membrane.

In some embodiments, the method further comprises administering acetate to the subject. In some embodiments, acetate is administered with citrate. In some embodiments, acetate is administered as an acetate salt or triacetin.

The invention provides a composition comprising citrate for use in a method of the invention.

The invention provides use of citrate to promote or maintain cell differentiation.

The invention provides use of citrate to promote histone acetylation.

The invention provides use of citrate to promote or maintain gene expression.

The invention provides a method of determining an optimal dosage of citrate in a subject for achieving a cosmetic or therapeutic effect, the method comprising determining the level of citrate in the subject and informing the subject whether their level of citrate is at, above or below a threshold level.

In some embodiments, the level of citrate is determined in a biological sample from the subject, e.g. a blood sample, serum sample, plasma sample, or urine sample.

In some embodiments, the method comprises recommending that the subject increases their intake of citrate when the level of citrate is below the threshold level.

In some embodiments, the cosmetic effect is selected from: (i) promoting hair growth; (ii) treating or preventing male pattern baldness; (iii) treating or preventing greying of hair; (iv) treating or preventing a condition associated with aging; (v) treating or preventing scar tissue; and (vi) improving the condition of skin.

In some embodiments, the therapeutic effect is selected from: (i) treating or preventing alopecia; (ii) treating or preventing a condition associated with aging; (iii) treating or preventing hearing loss; (iv) treating or preventing osteoporosis; (v) treating or preventing sarcopenia; (vi) promoting glucose homeostasis; (vii) treating or preventing diabetes; (viii) promoting wound healing in a subject; (ix) treating or preventing scar tissue; (x) improving kidney function; (xi) improving pancreatic function; and (xii) treating or preventing cancer.

### BRIEF DESCRIPTION OF FIGURES

FIG. 1. Images showing growth of hairs following administration of citrate on areas of the scalp that had suffered hair loss.

FIG. 2. Images showing growth of dark thicker hair following administration of an increased concentration of citrate.

FIG. 3. Image showing a comparison on the subject's scalp prior to administration of citrate (top photograph) and after administration of citrate (bottom photograph).

FIG. 4. Image showing a comparison on the subject's scalp prior to administration of citrate (top photograph) and after administration of citrate (bottom photograph).

FIG. 5. Graphs showing blood glucose concentration (mmol/L) over a 24 hour period measured using a continuous glucose monitor (CGM). (a)-(c) represent blood glucose levels in the absence of citrate administration; and (d) and (e) represent blood glucose levels following citrate administration.

FIG. 6. Images of facial blemishes (indicated by white lines within boxed area) prior to administration of citrate (top photograph) and after administration of citrate (bottom photograph).

FIG. 7. Image of a precancerous spot that was reduced in appearance following administration of citrate.

### DETAILED DESCRIPTION OF THE INVENTION

Endogenous citrate is produced in the mitochondria as part of the citric acid cycle. A portion of mitochondrial citrate is typically transported to the cytosol via citrate carrier proteins in the mitochondrial inner membrane. Some of this cytosolic citrate is then converted to acetyl-CoA which subsequently enters the nucleus where it plays an essential role in histone acetylation. Histone acetylation regulates gene expression. Upon histone acetylation, condensed chromatin is transformed into a more relaxed structure which enables transcription factors to more readily access DNA, leading to increased gene expression.

In arrested stem cells, expression of the citrate carrier protein is typically reduced, leading to reduced export of citrate from the mitochondria and a reduced concentration of cytosolic citrate. This in turn results in a shortage of acetyl-CoA which reduces or prevents histone acetylation. Reduced histone acetylation interferes with gene expression thereby reducing or preventing stem cell differentiation. Arrested stem cells also produce the cytokine interleukin-10 which results in the downregulation of nuclear factor kappa B (NFκB). NFκB is required for expression of the citrate carrier and so downregulation of NFκB further reduces export of citrate from the mitochondria to the cytosol, thereby further contributing to maintenance of cells in an arrested state.

Histone acetylation is also required for gene expression in differentiated cells. As noted, reduced cytosolic citrate results in reduced acetyl-CoA availability and reduced histone acetylation rates. In differentiated cells, reduced histone acetylation may result in the silencing of genes that are essential to the proper functioning of the cell and may eventually result in dedifferentiation of the cell.

Histone acetylation increases gene expression by opening the chromatin structure thereby increasing the accessibility of transcription machinery to DNA. Thus, histone acetylation is particularly important for longer genes which typically require a higher degree of histone acetylation as compared to shorter genes to ensure that the entire length of the gene is accessible by transcription machinery. As subjects age, the availability of acetyl-CoA in the nucleus and therefore the rate of histone acetylation decreases resulting in reduced expression of certain genes, e.g. long genes. The term “long genes” typically refers to genes which have a length in excess of 10,000 base pairs. Long genes are particularly subject to this reduced expression, and there is a gradual scale of reduced expression which links to gene length.

Without wishing to be bound by theory, the Inventor believes that administration of citrate increases the concentration of cytosolic citrate, thereby increasing histone acetylation via increased availability of acetyl-CoA. By ensuring that cells are able to effectively remodel chromatin when necessary, increased histone acetylation promotes cell differentiation and also maintains cells in a (functional) differentiated state. In addition, increased acetyl-CoA availability maintains and promotes gene expression, particularly gene expression that is heavily reliant on histone acetylation.

Using the differentiation of cells required for hair growth as a model system, the Inventor found that administration of citrate unexpectedly resulted in the growth of new hairs on areas of the scalp which had suffered hair loss up to 20 years previously. The Inventor believes that administration of citrate achieved this remarkable effect by promoting differentiation of hair follicle stem cells which have been arrested in a quiescent state for a number of years. The Inventor also believes that administration of citrate maintained cells that are required for hair growth in a differentiated state, thereby reducing or preventing further hair loss.

The Inventor also discovered that topical administration of red or infrared radiation with a wavelength of between 600 and 900 nanometres in addition to citrate administration further improved hair growth. The Inventor believes that red or infrared radiation improves mitochondrial efficiency by resonating the copper atoms in Cytochrome C oxidase. Thus, administration of citrate and red or infrared radiation function together to increase acetyl-CoA production.

A further unexpected result was that administration of citrate resulted in an increase in muscle mass which exceeded any muscle mass increase that might be associated with limited exercise. The Inventor believes that the observed increase in muscle mass is a result of increased differentiation of muscle stem cells.

The results described herein demonstrate that administration of citrate promotes the differentiation of stem cells, as demonstrated for muscle and hair follicle cells. Reduced stem cell differentiation plays a key role in various inflammatory diseases (such as atherosclerosis) and musculoskeletal diseases (such as osteoporosis) and the Inventor believes that the methods described herein would be equally effective at treating or preventing these conditions.

Similarly, various conditions associated with aging involve reduced cell differentiation and/or increased cell dedifferentiation. The ability of citrate to promote and maintain cell differentiation enables the methods described herein to be used in the treatment of these conditions.

Wound healing relies on the differentiation and proper functioning of cells. The data described herein demonstrate that administration of citrate advantageously promotes and maintains cell differentiation and also supports normal cell functioning thereby promoting wound healing.

The invention provides a method of promoting or maintaining cell differentiation in a subject, the method comprising administering citrate to the subject. The invention also provides citrate for use in a method of promoting or maintaining cell differentiation in a subject. Administration of citrate increases the concentration of cytosolic citrate, which results in increased availability of acetyl-CoA. Acetyl-CoA is an essential component for histone acetylation, which is itself essential for enabling changes in gene expression required for cell differentiation. Histone acetylation is also required for normal gene expression within cells. When histone acetylation is reduced (e.g. in response to an acetyl-CoA deficit), the chromatin structure cannot readily unwind, thereby reducing or inhibiting the ability of transcription factors to access DNA. As a result, reduced levels of gene expression occur and gene silencing may result. Reduced histone acetylation levels may eventually lead to dedifferentiation of cells. Thus, by providing cells with citrate (and therefore acetyl-CoA), histone acetylation is supported, and cells are maintained in a differentiated state.

A shortage of Acetyl-CoA negatively impacts the activity of the RNA Polymerase II complex and may result in gene transcription stalling and/or terminating prematurely. This is particularly problematic for the expression of longer genes (e.g. genes that are ≥10,000 base pairs) which are more likely to be inaccessible to transcription factors via inefficient chromatic remodelling. If the Acetyl-CoA shortage persists then the transcription of the gene into messenger RNA terminates and no protein is produced. As noted, acetyl-CoA deficit is more likely to result in reduced gene expression and reduced production of proteins from longer genes. In addition, long genes are likely to suffer from reduced transcription as cells age. Thus, administration of citrate can increase the expression of genes, particularly long genes, by increasing the availability of acetyl-CoA and this can advantageously reduce and/or prevent an effect of aging as described herein.

Administration of a histone deacetylase inhibitor reduces the likelihood that a stalled RNA Polymerase II complex will terminate transcription prematurely, thereby increasing gene expression.

Any process which encourages autophagy will operate synergistically with administration of citrate. Autophagy, and in particular mitophagy, is the process by which cells recycle mitochondria and become more efficient. This has the effect of increasing the production both of ATP and of citrate in the cytosol. The protein beclin-1 is a key protein for the process of autophagy, but its gene BECLN1 is a long gene which is less likely to be expressed with a shortage of nuclear acetyl-CoA. Hence, taking exogenous citrate which is available to the cells when autophagy is upregulated makes autophagy more likely to occur.

As used herein, “promoting cell differentiation” means increasing the likelihood that cells (e.g. stem cells) will undergo differentiation in response to appropriate stimuli to produce specialised (differentiated) cell types, as compared to cells in the absence of citrate administration. In some embodiments, the cells are arrested in a quiescent state prior to differentiation (also referred to herein as “arrested cells”). In some embodiments, the method comprises promoting differentiation of undifferentiated cells. In some embodiments, the method comprises promoting differentiation of stem cells. In some embodiments, the method comprises promoting differentiation of dedifferentiated cells.

As used herein, “maintaining cell differentiation” means decreasing the likelihood that cells will revert to a less differentiated stage within the cell lineage, as compared to cells in the absence of citrate administration. In the context of the invention, cell dedifferentiation is associated with disruption of gene expression, as a result of reduced levels of histone acetylation. As used herein, maintaining cell differentiation encompasses maintaining cell function. By ensuring that cells can maintain effective histone acetylation levels, cells are maintained in a differentiated state, and optimal cell function (e.g. optimal gene expression) is maintained.

The invention also provides a method of promoting histone acetylation in a subject, the method comprising administering citrate to the subject. The invention also provides citrate for use in a method of promoting histone acetylation in a subject. As noted, administration of citrate increases the concentration of cytosolic citrate which leads to increased availability of acetyl-CoA. Acetyl-CoA is essential for histone acetylation, and so increasing the availability of acetyl-CoA increases the rate of histone acetylation within cells. In differentiated cells, increased (or maintained) histone acetylation helps ensure that cells function properly and do not undergo dedifferentiation. Increased histone acetylation may also increase gene expression, thereby enabling cells to increase their functional abilities. Increasing histone acetylation is a means for improving cell health. Thus, in some embodiments, the method comprises improving cell health in the subject. In some embodiments, improving cell health comprises improving the health of at least one of hair follicle cells, muscle cells, skin cells, or stem cells.

In some embodiments, the method comprises administering citrate to the subject in combination with an antioxidant. The administration of citrate may result in increased production of reactive oxygen species (ROS) which can lead to negative physiological effects, such as increased inflammatory responses. Advantageously, administration of citrate in combination with an antioxidant helps mitigate the negative effects associated with increased production of ROS. In some embodiments, the antioxidant is selected from melatonin, ethanol, a carotenoid, a flavonoid, ascorbic acid, vitamin E, and grape seed extract. In some embodiments, the antioxidant is administered before, simultaneously with, or after the administration of citrate.

In some embodiments, the antioxidant is melatonin. Without wishing to be bound by theory, the Inventor believes that there is a synergistic effect when citrate is administered in combination with melatonin. This is because melatonin increases the efficiency of mitochondria which further improves cellular efficiency and citrate production and uptake.

In some embodiments, the method comprises administering citrate to the subject in combination with an anti-inflammatory agent. Advantageously, administration of citrate in combination with an anti-inflammatory agent reduces unwanted inflammatory effects that may be associated with citrate administration. In some embodiments, the anti-inflammatory agent is a non-steroidal anti-inflammatory drug (e.g. aspirin or ibuprofen). In some embodiments, the anti-inflammatory agent is a corticosteroid. In some embodiments, the anti-inflammatory agent is an antioxidant. In some embodiments, the anti-inflammatory agent is administered before, simultaneously with, or after the administration of citrate.

In some embodiments, the method comprises administering multiple doses of citrate to the subject. In some embodiments, the method comprises administering multiple doses of citrate to the subject during a 24 hour period. Citrate is rapidly cleared from the blood stream and so administering multiple doses throughout the day ensures that an effective level of citrate is maintained in the blood stream for a longer period of time. In some embodiments, the method comprises administering citrate to the subject at least 2 times per day. In some embodiments, the method comprises administering citrate to the subject at least 3 times, at least 4 times, at least 5 times, or at least 6 times per day. In some embodiments, the method comprises administering citrate to the subject 3 times a day, e.g. with breakfast, lunch and dinner. In some embodiments, the method comprises administering citrate to the subject at intervals of at least 2 hours, at least 3 hours, at least 4 hours, at least 6 hours, or at least 12 hours.

Wherein the citrate is administered in combination with an antioxidant and/or an anti-inflammatory agent, some embodiments comprise administering the antioxidant and/or the anti-inflammatory agent with each dose of citrate. In some embodiments a single dose of the antioxidant and/or the anti-inflammatory agent is administered to the subject per day. In some embodiments, the method comprises administering at least two, at least 3, or at least 6 doses of the antioxidant and/or the anti-inflammatory agent in a 24 hour period.

Wherein the antioxidant is melatonin, some embodiments of the method comprise administering a single dose of melatonin to the subject. Melatonin may be taken up by cells and stored in the mitochondria and so a single dose of melatonin may advantageously provide a prolonged antioxidant effect. In some embodiments, melatonin is administered to the subject during sleep to avoid or minimise interference with sleep wake cycles. In some embodiments, the method comprises administering a time release composition comprising melatonin to the subject. In some embodiments, melatonin is released from the time-release composition at least 1 hour after administration of the composition to the subject. In some embodiments, melatonin is released from the time-release composition at least 2 hours, at least 3 hours, at least 4 hours, at least 5 hours, or are least 6 hours after administration of the composition to the subject.

In some embodiments, the cells are stem cells. In some embodiments, the stem cells are hair follicle stem cells. In some embodiments, the stem cells are mesenchymal stem cells. In some embodiments, the stem cells are muscle stem cells.

In some embodiments, the method comprises promoting and/or maintaining the differentiation of cells required for hair growth, e.g. hair follicle cells and/or hair follicle stem cells. Promoting the differentiation of hair follicle stem cells, particularly the differentiation of hair follicle stem cells which are arrested in a quiescent state, results in the production of new hair strands, even in areas of the scalp which have suffered hair loss. Moreover, maintaining hair follicle cells in a differentiated state ensures that hair follicles remain active, thereby allowing the continued production of hair strands and reducing or preventing hair loss. Thus, in some embodiments, the method comprises promoting hair growth in a subject and/or preventing hair loss in a subject.

In some embodiments, the method comprises treating or preventing alopecia. The invention also provides a composition comprising citrate for use in a method of treating or preventing alopecia.

In some embodiments, the method comprises treating or preventing male pattern baldness (androgenic alopecia). The invention also provides a composition comprising citrate for use in a method of treating or preventing male pattern baldness.

In some embodiments, the method comprises treating or preventing greying of hair. Maintaining hair follicle cells in a differentiated state ensures that these cells continue to function properly, which may advantageously prevent pigment loss associated with the production of grey hairs. Unexpectedly, the Inventor discovered that increased levels of citrate increased the production of dark hairs. Wherein the method comprises treating or preventing greying of hair, some embodiments of the method comprise administering multiple doses of citrate to the subject in a 24 hour period. Wherein the method comprises treating or preventing greying of hair, some embodiments of the method comprise administering at least 3 g of citrate to the subject, optionally, at least 4 g, at least 5 g, at least 6 g, at least 7 g, at least 8 g, at least 9 g, or at least 10 g of citrate.

Unless otherwise stated, the weight of citrate refers to the weight of citrate anion.

In some embodiments, the method comprises treating or preventing hearing loss. Changes to hearing typically occur when hair cells within the inner ear are lost or damaged. Promoting the differentiation of stem cells to produce hair cells and/or maintaining hair cells in a differentiated state may help to prevent and/or treat hearing loss. As noted, maintaining cells in a differentiated state helps ensure that cells retain their functional abilities, and so the methods of the invention may advantageously increase the functioning of existing hair cells, e.g. damaged hair cells. The invention also provides a composition comprising citrate for use in a method of treating or preventing hearing loss.

In some embodiments, the method comprises treating or preventing osteoporosis. By promoting differentiation of mesenchymal stem cells, the invention provides a method of increasing the production of osteoblasts which may help to rebalance the bone regeneration process, thereby preventing or treating osteoporosis. The invention also provides a composition comprising citrate for use in a method of treating or preventing osteoporosis.

In some embodiments, the method comprises treating or preventing sarcopenia. Unexpectedly, the Inventor has found that the method of the invention promotes an increase in muscle mass, indicating that differentiation of muscle stem cells (satellite cells) has been promoted. The invention also provides a composition comprising citrate for use in a method of treating or preventing sarcopenia.

In some embodiments, the method comprises treating or preventing atherosclerosis. The invention also provides a composition comprising citrate for use in a method of treating or preventing atherosclerosis.

In some embodiments, the method comprises promoting or improving glucose homeostasis in a subject. Glucose homeostasis is the process by which the level of glucose dissolved in blood plasma is maintained within a narrow concentration range, typically about 4 mM to about 6 mM. The primary regulators of glucose homeostasis are insulin, which acts to reduce blood glucose levels, and glucagon, which acts to increase blood glucose levels. As used herein, promotion or improvement of glucose homeostasis means that blood glucose levels are more likely to remain within a normal concentration range, and the occurrence of spikes oh high or low blood glucose levels (outside the normal concentration range) is reduced. The Inventor has surprisingly found that administration of citrate improves glucose homeostasis, and reduces the occurrence of high blood glucose. The Inventor believes that this is achieved by promoting and/or maintaining the expression of longer genes in pancreatic cells (by avoiding an acetyl-CoA deficit in these cells) thereby preventing them becoming adipocytes. Advantageously, properly functioning pancreatic cells can continue to generate insulin and avoid absorbing lipids to generate fat and block other cells from producing insulin, thereby promoting and improving glucose homeostasis, and reducing the likelihood that the subject will develop type 2 diabetes.

In some embodiments, the method comprises treating or preventing type 2 diabetes. Type 2 diabetes is characterised by high blood glucose levels, insulin resistance and reduced insulin production. Advantageously, the Inventor has discovered that administration of citrate promotes normal glucose homeostasis and can therefore treat or prevent the onset of type 2 diabetes.

In some embodiments, the method comprises promoting wound healing in a subject. Promoting the differentiation of stem cells advantageously increases the rate of tissue cell (e.g. skin cell) production thereby promoting wound healing. In addition, maintaining cells in a differentiated state ensures that cells required for wound healing can function efficiently. The invention also provides a composition comprising citrate for use in a method of promoting wound healing.

In some embodiments, the method comprises preventing or treating scar tissue. By maintaining cells in a differentiated state and promoting the differentiation of stem cells, tissue (e.g. skin) regeneration is promoted thereby reducing or preventing the formation of scar tissue.

In some embodiments, the method comprises improving the condition of the skin. In some embodiments, the method is a cosmetic method for improving the condition of the skin. In some embodiments, improving the condition of the skin comprises reducing the appearance of wrinkles, increasing firmness, and/or reducing the appearance of scars. By promoting the regeneration of skin tissue, the methods of the invention advantageously improve the condition and appearance of skin.

In some embodiments, the method comprises improving kidney function. In some embodiments, the method comprises treating or preventing kidney function loss. Kidney function loss may be associated with decreased functionality of kidney cells (e.g. hepatocytes) as a result of decreased gene expression or dedifferentiation. By promoting and/or maintaining cell differentiation and gene expression, the methods of the invention improve kidney function.

In some embodiments, the method comprises improving pancreatic function. In some embodiments, the method comprises treating or preventing pancreatic function loss. Pancreatic function loss may be associated decreased functionality of pancreatic cells as a result of decreased gene expression or dedifferentiation. By promoting and/or maintaining cell differentiation and gene expression, the methods of the invention improve pancreatic function.

In some embodiments, the method comprises treating or preventing a condition associated with aging. Dysregulation of cell differentiation is more likely to occur as cells grow older and so promoting and/or maintaining cell differentiation may advantageously treat and/or prevent conditions associated with aging. In some embodiments, the condition associated with aging is reduced skin integrity (e.g. increased presence of wrinkles), or hair loss. In some embodiments, the condition associated with aging is atherosclerosis, osteoporosis, or sarcopenia.

In some embodiments, the method comprises administering citrate to the subject in combination with an anti-cholesterol agent. Administration of an anti-cholesterol agent advantageously counteracts any potential increase in cholesterol levels associated with increased levels of cytosolic citrate. In some embodiments, the anti-cholesterol agent is a statin. In some embodiments, the method comprises monitoring cholesterol levels in a biological sample obtained from the subject, and administering the anti-cholesterol agent if cholesterol levels exceed a threshold level. Suitable cholesterol threshold levels for the subject's age, and methods for detecting cholesterol levels are well known in the art. The anti-cholesterol agent may be administered using any suitable method known in the art.

In some embodiments, the citrate is administered as citric acid. In some embodiments, the method comprises administering to the subject citrate in the form of lemon juice. Lemon juice typically comprises approximately 0.05 g citrate per ml. In some embodiments, the method comprises administering to the subject at least 5 ml, at least 10 ml, at least 15 ml, at least 20 ml, or at least 25 ml lemon juice per day. In some embodiments, the method comprises administering one or more types of citric acid containing fruits to the subject, e.g. limes, lemons, oranges, grapefruit, or blackberries.

In some embodiments, the citrate is administered as a salt. In some embodiments, the salt comprises a cation selected from calcium, magnesium, potassium, and sodium.

In some embodiments, citrate is administered as a combination of a salt and citric acid.

In some embodiments, the method comprises administering to the subject at least 0.5 g of citrate per day. In some embodiments, the method comprises administering to the subject at least 1.85 g, at least 2 g, at least 3 g, at least 4 g, at least 5 g, at least 6 g, at least 7 g, at least 8 g, at least 9 g, at least 10 g, at least 15 g, at least 20 g, at least 25 g, or at least 30 g of citrate per day.

In some embodiments, the method comprises administering to the subject up to 2 g, up to 3 g, up to 4 g, up to 5 g, up to 6 g, up to 7 g, up to 8 g, up to 9 g, up to 10 g, up to 15 g, up to 20 g, up to 25 g, up to 29 g, or up to 30 g of citrate per day.

In some embodiments, the method comprises administering to the subject 2-30 g, 3-30 g, 4-30 g, 5-30 g, 10-30 g, 15-30 g, 20-30 g, 25-30 g, 2-25 g, 3-25 g, 4-25 g, 5-25 g, 10-25 g, 15-25 g, or 20-25 g of citrate per day.

Typically, the method comprises administering to the subject at least 1.85 g of citrate per day. Preferably, the method comprises administering to the subject at least 4 g of citrate per day. In some embodiments, the method comprises administering to the subject at least 6 g of citrate per day.

In some embodiments, the method comprises administering to the subject at least 1.85 g of citrate per day for four or more weeks. In some embodiments, the method comprises administering to the subject at least 4 g of citrate per day for four or more weeks. In some embodiments, the method comprises administering to the subject at least 6 g of citrate per day for four or more weeks.

In some embodiments, the method comprises administering to the subject at least 1.85 g of citrate per day for eight or more weeks. In some embodiments, the method comprises administering to the subject at least 4 g of citrate per day for eight or more weeks. In some embodiments, the method comprises administering to the subject at least 6 g of citrate per day for eight or more weeks.

In some embodiments, the method comprises administering to the subject at least 3 g of citrate per day for 3 months or more.

In some embodiments, repeated doses of citrate and acetate are administered to the subject throughout a 4 hour, 6 hour, 8 hour, 10 hour, 12 hour, 24 hour period, or 48 hour period. Acetate may be administered as a salt or as triacetin. In some embodiments, citrate is administered with a compound that is metabolised to produce acetate.

In some embodiments, citrate is administered to subject wherein the subject has ingested ethanol. In some embodiments, repeated doses of citrate are administered to the subject throughout a 4 hour, 6 hour, 8 hour, 10 hour, 12 hour, 24 hour period, or 48 hour period wherein the subject has ingested ethanol at least once during said period.

In some embodiments, wherein repeated doses of citrate are administered to the subject, the method comprises administering two or more doses of citrate in a given time period, e.g. three or more, four or more, five or more, six or more, or 10 or more.

Wherein the citrate is administered as a salt, some embodiments of the method comprise: (i) monitoring the level of at least one cation of the citrate salt in a biological sample obtained from the subject; and (ii) stopping or pausing citrate administration if the level of said at least one cation of the citrate salt exceeds a threshold level; optionally wherein the cation is selected from calcium, magnesium, potassium, and sodium. In some embodiments, the biological sample is selected from a blood sample, serum sample, plasma sample, or urine sample. Cation levels may be determined using any suitable method known in the art, e.g. using photometric methods or atomic absorption spectrometry.

In some embodiments, the method comprises: (i) determining the pH of a biological sample obtained from the subject; and (ii) stopping or pausing citrate administration and/or administering a substance to reduce pH if the pH level exceeds a threshold level. In some embodiments, biological samples are obtained from the subject for pH determination every day, every 2 days, every 4 days, every week, every 2 weeks, or every 4 weeks. In some embodiments, the biological sample is blood. In some embodiments, the biological sample is blood and the threshold pH level is 8.5, optionally wherein the threshold pH is 9, 9.5, 10 or 10.5. In some embodiments, the biological sample is serum. In some embodiments, the biological sample is serum and the threshold pH level is 8.5, optionally wherein the threshold pH is 9, 9.5, 10 or 10.5. In some embodiments, the biological sample if urine. In some embodiments, the biological sample is urine and the threshold pH level is 8.5, optionally wherein the threshold pH is 9, 9.5, 10 or 10.5.

Cells use histone acetylation and deacetylation to regulate intracellular pH, e.g. when exposed to acidic environments, cells induce histone deacetylation to increase alkalinity. Thus, by maintaining an alkaline pH within the body, cells remain in an alkaline state and histone deacetylation is avoided allowing cells to continue functioning optimally. In some embodiments, the method comprises: (i) determining the pH of a biological sample obtained from the subject; and (ii) if the pH is below a threshold level, recommending dietary adjustments to increase the pH level. In some embodiments, biological samples are obtained from the subject for pH determination every day, every 2 days, every 4 days, every week, every 2 weeks, or every 4 weeks. In some embodiments, the biological sample is urine and the threshold pH level is 7. In some embodiments, the biological sample is blood and the threshold pH level is 7.4. In some embodiments, the biological sample is serum and the threshold pH level is 7.4. In some embodiments, dietary adjustments comprise reducing consumption of foods with a high potential renal acid load, e.g. meat, fish eggs, and dairy products.

In some embodiments, the method comprises administering at least 1 g of citrate per dose to the subject, optionally, at least 1.1 g, at least 1.2 g, at least 1.3 g, at least 1.4 g, at least 1.5 g, at least 1.6 g, at least 1.7 g, at least 1.8 g, at least 1.85 g, at least 1.9 g, at least 2 g, at least 3 g, at least 3.5 g, at least 3.95 g, at least 4 g, at least 5 g, at least 6 g, at least 7 g, at least 8 g, at least 9 g, or at least 10 g of citrate per dose.

In some embodiments, the method comprises: (i) monitoring inflammation levels in the subject; and (ii) stopping or pausing citrate administration if inflammation levels exceed a threshold level. In some embodiments, monitoring inflammation levels comprises detecting the level of an inflammation marker in a biological sample obtained from the subject. In some embodiments, the biological sample is selected from a blood sample, a serum sample, or a plasma sample. In some embodiments, monitoring inflammation levels comprises measuring the erythrocyte sedimentation rate (ESR) in a blood sample obtained from the subject. The ESR is determined by the rate at which red blood cells sink to the bottom of e.g. a test tube. A high ESR indicates increased levels of inflammation.

In some embodiments, the method comprises administering citrate to the subject in combination with one or more supplements selected from vitamin D3, boron, zinc, quercetin, β-Nicotinamide mononucleotide (NMN), nicotinamide riboside (NR), sulforaphane, chia seeds, black turkey beans, psyllium husk, hyaluronic acid, berberine, ferulic acid, phosphorylated serine, 25 hydroxyvitamin G (25 (OH) D), aspirin, resveratrol, pterostilbene, pantethine, ketone ester, medium chain triglycerides (MCT), vitamin A, vitamin B, vitamin C, vitamin E or vitamin K (including vitamin K2 MK4, vitamin K2 MK7 and vitamin K2 MK9), fish oil, collagen, curcumin, carnosine, betaine, astaxanthin and grape seed extract.

In some embodiments, the method comprises administering citrate to the subject in combination with molecular hydrogen.

In some embodiments, the method comprises administering citrate to the subject if the level of C reactive protein (CRP) in a biological sample obtained from the subject exceeds a threshold level. In this embodiment, the level of CRP is typically measured prior to administration of citrate. A high level of CRP may be indicative of a high burden of senescent cells leading to a high level of senescence-induced inflammation. The Inventor has advantageously found that administration of citrate reduces CRP levels in the blood. Without wishing to be bound by theory, the Inventor believes that administration of citrate advantageously promotes the differentiation of senescent cells and prevents differentiated cells from becoming senescent, thereby decreasing the senescent cell burden in the subject.

The invention provides a composition comprising citrate for use in a method of the invention. In some embodiments, the composition is formulated as a time-release composition and/or a sustained release composition.

The invention also provides use of citrate to promote histone acetylation. As described herein, administration of citrate increases the concentration of cytosolic citrate which increases the availability of acetyl-CoA. Increased availability of acetyl-CoA allows cells to maintain a high rate of histone acetylation. The invention also provides a composition comprising citrate for use in a method of promoting histone acetylation.

The invention also provides use of citrate to promote and maintain cell differentiation. The invention also provides a composition comprising citrate for use in a method of promoting and maintaining cell differentiation. Administration of citrate increases the concentration of cytosolic citrate which results in increased availability of acetyl-CoA. Acetyl-CoA is required for histone acetylation, which is itself essential for enabling the changes in gene expression required for cell differentiation. Histone acetylation is also required for normal gene expression within cells. When histone acetylation is reduced (e.g. in response to an acetyl-CoA deficit), the chromatin structure cannot readily unwind, thereby inhibiting the ability of transcription factors to access DNA. As a result, reduced levels of gene expression occur and gene silencing may result. Reduced histone acetylation levels may eventually lead to dedifferentiation of cells. Thus, by providing cells with citrate (and therefore acetyl-CoA), histone acetylation is supported, and cells are maintained in a differentiated state.

The invention also provides a method of determining an optimal dosage of citrate in a subject for achieving a cosmetic or therapeutic effect, the method comprising determining the level of citrate in the subject and informing the subject whether their level of citrate is at, above or below a threshold level. The level of citrate may be measured in a biological sample from the subject, e.g. a blood sample, serum sample, plasma sample, or urine sample. In some embodiments, the biological sample is blood or urine. In some embodiments, the method comprises recommending that the subject increases their intake of citrate when the level of citrate is below the threshold level.

In some embodiments, the cosmetic effect is selected from: (i) promoting hair growth; (ii) treating or preventing male pattern baldness; (iii) treating or preventing greying of hair; and (iv) treating or preventing a condition associated with aging.

In some embodiments, the therapeutic effect is selected from: (i) treating or preventing alopecia; (ii) treating or preventing a condition associated with aging; (iii) treating or preventing hearing loss; (iv) treating or preventing osteoporosis; (v) treating or preventing sarcopenia; (vi) promoting glucose homeostasis; (vii) treating or preventing diabetes; (viii) promoting wound healing in a subject; (ix) treating or preventing scar tissue; (x) improving kidney function; (xi) improving pancreatic function; and (xii) treating or preventing cancer.

The invention also provides a method of promoting telomere lengthening and/or reducing or preventing telomere shortening, the method comprising administering citrate to a subject. Telomerase reverse transcriptase (hTERT) is required to lengthen telomeres and expression of hTERT requires hyperacetylation of histones. Thus, by ensuring that sufficient acetyl-CoA is available to support histone acetylation the invention advantageously ensures that hTERT can be expressed efficiently.

The citrate may be formulated for any suitable route of administration. In some embodiments, the citrate is administered systemically. In some embodiments, the citrate is administered to the subject by sublingual administration, intravenous administration, transdermal administration, or oral administration. In some embodiments, the citrate is formulated for sublingual administration, intravenous administration, oral administration, transdermal administration, subcutaneous administration, intraperitoneal administration, mucosal administration, or rectal administration.

The antioxidant may be formulated for any suitable route of administration. In some embodiments, the antioxidant is administered systemically. In some embodiments, the antioxidant is formulated for sublingual administration, intravenous administration, oral administration, transdermal administration, subcutaneous administration, intraperitoneal administration, mucosal administration, or rectal administration. In some embodiments, the antioxidant is administered by sublingual administration, intravenous administration, transdermal administration, or oral administration.

The anti-inflammatory agent may be formulated for any suitable route of administration. In some embodiments, the anti-inflammatory agent is administered systemically. In some embodiments, the anti-inflammatory agent is formulated for sublingual administration, intravenous administration, oral administration, transdermal administration, subcutaneous administration, intraperitoneal administration, mucosal administration, or rectal administration. In some embodiments, the anti-inflammatory agent is administered by sublingual administration, intravenous administration, transdermal administration, or oral administration.

In some embodiments, the citrate is formulated as a sustained release composition comprising citrate. In some embodiments, the sustained release composition is formulated to release citrate over a predetermined period of time. In some embodiments, the sustained release composition is formulated to release citrate over a period of at least 2 hours, at least 3 hours, at least 4 hours, at least 5 hours, at least 6 hours, at least 8 hours, at least 10 hours, at least 12 hours, or at least 24 hours. Advantageously, sustained release compositions ensure that continued dosing of citrate is achieved over a period of time thereby ensuring that an effective amount of citrate is maintained for a prolonged period.

In some embodiments, the citrate is formulated as a time-release composition comprising citrate. In some embodiments, the time-release composition is formulated to release citrate after a predetermined period of time. In some embodiments, the time-release composition is formulated to release citrate after a period of at least 1 hour, at least 2 hours, at least 3 hours, at least 4 hours, at least 5 hours, at least 6 hours, at least 8 hours, at least 10 hours, or at least 12 hours. In some embodiments, the time-release composition is formulated for sustained release as described herein.

The citrate may be combined or administered with a carrier, diluent and/or excipient. Generally, the carrier is a pharmaceutically-acceptable carrier. Non-limiting examples of pharmaceutically acceptable carriers include water, saline, and phosphate-buffered saline. In some embodiments, the citrate may be in lyophilized form, in which case it may include a stabilizer, such as BSA. In some embodiments, it may be desirable to formulate the citrate with a preservative, such as thiomersal or sodium azide, to facilitate long term storage.

The citrate may be prepared as an injectable, either as a liquid solution or suspension. Solid forms suitable for solution in, or suspension in, liquid prior to injection may alternatively be prepared. The citrate may be encapsulated or embedded in a delivery vehicle. In various aspects, the delivery vehicle is a liposome, a lysosome, a microcapsule, or a nanoparticle. Oral compositions may include normally employed excipients such as pharmaceutical grades of mannitol, lactose, starch, magnesium stearate, sodium saccharine, cellulose, magnesium carbonate, and the like. These compositions take the form of solutions, suspensions, tablets, pills, capsules, sustained release compositions or powders.

In some embodiments, the citrate is formulated for topical administration. In some embodiments, the citrate is formulated as a liquid, spray or aerosol. In some embodiments, the topical formulation comprises a solvent. Suitable solvents include, but are not limited to, DMSO, Propylene glycol, Dipropylene, and propanol. The presence of a solvent advantageously encourages absorption of citrate across the cell membrane.

The subject is typically a human subject. In some embodiments, the subject has been identified as having hair loss, optionally wherein the subject has been identified as having alopecia or male pattern baldness. In some embodiments, the subject has been identified as having atherosclerosis. In some embodiments, the subject has been identified as having hearing loss. In some embodiments, the subject has been identified as having osteoporosis. In some embodiments, the subject has been identified as having sarcopenia. In some embodiments, the subject has been identified as having insulin resistance. In some embodiments, the subject has been identified as being prediabetic. In some embodiments, the subject has been identified as having type II diabetes.

### EXAMPLES

The invention will be further clarified by the following examples, which are intended to be purely exemplary of the invention and are in no way limiting.

### Example 1

Citrate was administered to a subject in the morning as magnesium and potassium citrate and at lunch time as 10 ml of lemon juice to provide a total daily citrate dose of 1.85 g. The antioxidant melatonin was administered at night to provide a total nightly dose of between 10 mg and 470 mg. Citrate was administered daily for four weeks.

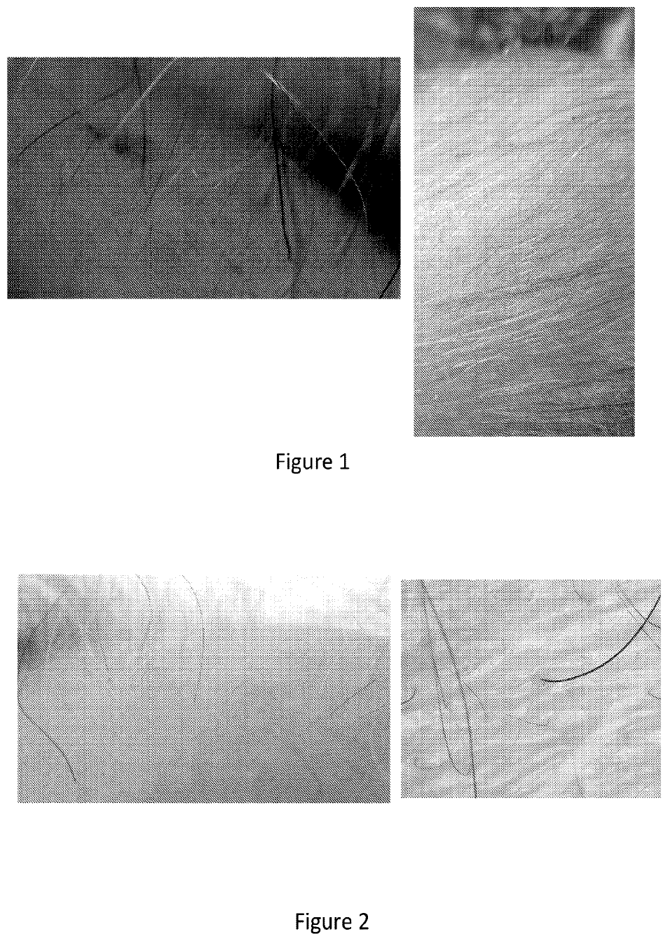

Following administration of citrate, the growth of new hairs was observed in areas of the scalp which had suffered hair loss up to 20 years previously (see FIG. 1). The new hairs that were observed were typically white and thin. The Inventor believes that the growth of new hairs is a result of increased hair follicle stem cell differentiation which resulted in the production of new hair strands by hair follicles which had been inactive for a number of years. These results indicate that administration of citrate can promote differentiation in stem cells which have been arrested in a quiescent state, even for a number of years.

### Example 2

Citrate was administered to the subject in the morning as magnesium and potassium citrate, at lunch time as 10 ml of lemon juice and as calcium and sodium citrate with a total daily citrate dose of 3.95 g. The antioxidant melatonin was administered at night to provide a total nightly dose of between 10 mg and 470 mg.

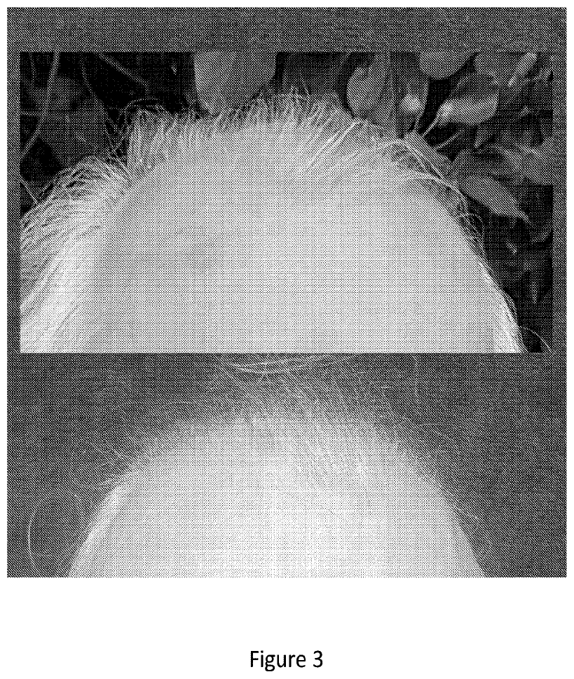

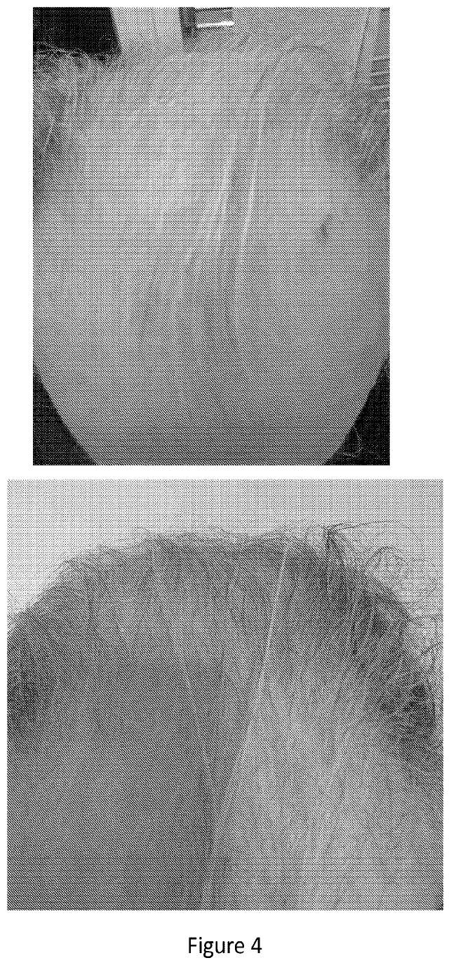

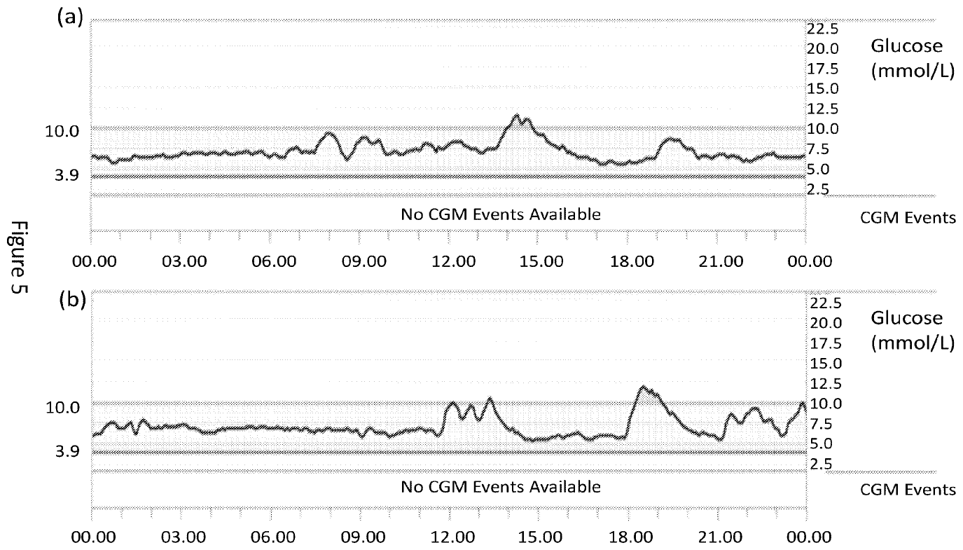

Surprisingly, increasing the dose of citrate (as compared to Example 1) resulted in the growth of hairs which were thicker and darker than the thin grey/white hairs that were observed in Example 1 (see FIGS. 2-4). The Inventor believes that increased production of dark hairs is associated with increased and/or maintained cell functioning and decreased cell dedifferentiation, as a result of increased cytosolic citrate availability.

An increase in muscle mass was also observed following administration of citrate. The Inventor believes that increased muscle mass results from enhanced differentiation of satellite cells. Importantly, these results indicate that administration of citrate may help to counteract muscle loss, e.g. associated with sarcopenia.

Administration of citrate also resulted in reduced CRP levels in the blood of the subject to below detectable levels. CRP levels were reduced from 0.5 mg/L to <0.3 mg/L (0.3 mg/L represents the lower limit of detection) following two months of citrate administration. A subsequent measurement of CRP using equipment with higher sensitivity identified a CRP level of below 0.16 mg/L. The Inventor believes that reduced CRP levels result from increased differentiation of senescent stem cells and reduced production of senescent cells via cell dedifferentiation.

### Example 3

Citrate was administered to the subject with a total daily citrate dose of between 2 and 40 g for a period of 10 months. Blood glucose levels were measured over separate 24 hour periods using a continuous glucose monitor (CGM) purchased from Dexcom.

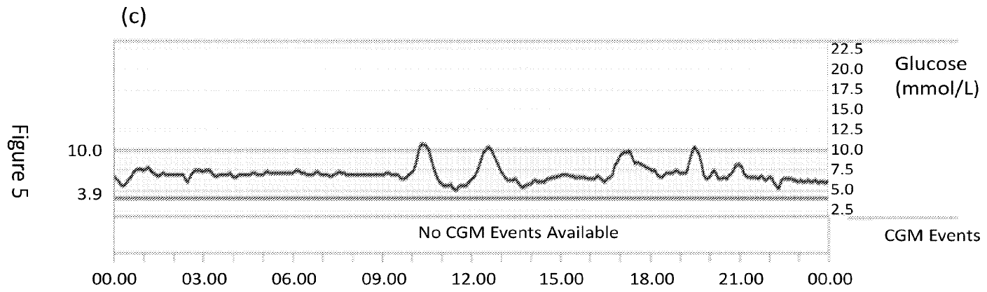

FIGS. 5(a)-(c) provide exemplary glucose levels from three non-consecutive days prior to citrate administration. The occurrence of peaks in glucose concentration throughout the day indicate a delay in glucose homeostasis mechanisms in response to food intake. In contrast, FIGS. 5(d) and (e) provide glucose levels from two non-consecutive days following administration of citrate. Surprisingly, no significant glucose peaks occurred across these time periods, despite the subject consuming similar foodstuffs as in FIGS. 5(a)-(c). These results indicate that blood glucose levels reached a lower peak level following citrate administration, suggesting improved glucose homeostasis.

The Inventor also measured haemoglobin A1C (HbA1c) which can be used as a diagnostic marker for diabetes. An HbA1c concentration of >6.4% is indicative of diabetes, and a concentration of 5.7% to 6.4% is indicative of prediabetes. Administration of citrate was found to maintain HbA1c levels at less than 5.7%, and even as low as 4.18%.

TABLE 1
Concentration of key biomarkers in the blood
following 6 months of citrate administration.

| | Numerical result | | Lower | Upper |
|---|---|---|---|---|
| Folate | 15.24 | ug/l | 2.5 | 20 |
| HbA1C | 4.18% | | 4 | 6 |
| Glucose | 4.53 | mmol/l | 3.3 | 5.6 |

### Example 4

The Inventor also found that administration of citrate at a total daily citrate dose of between 2 and 40 g for a period of 10 months improved kidney function. The subject exhibited reduced levels of Cystatin C from 0.98 mg/L to 0.89 mg/L following 3 months of citrate administration which implies an improvement to kidney function to close to that seen in the age range of 20-50 (which is 0.85 mg/L). The subject's creatinine levels reached as low as 62 mmol/L.

### Example 5

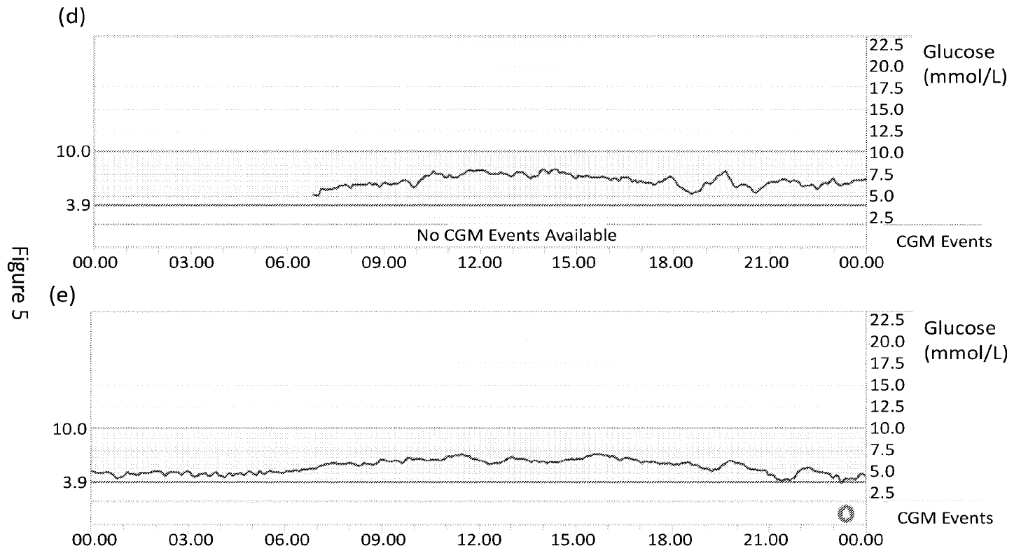

Administration of citrate was also found to improve facial skin quality following administration of between 2 and 40 g of citrate daily for a period of 6 weeks. FIG. 6 demonstrates a reduction in the number of blemishes from 25 to 20 (as indicated by white lines within boxed area).

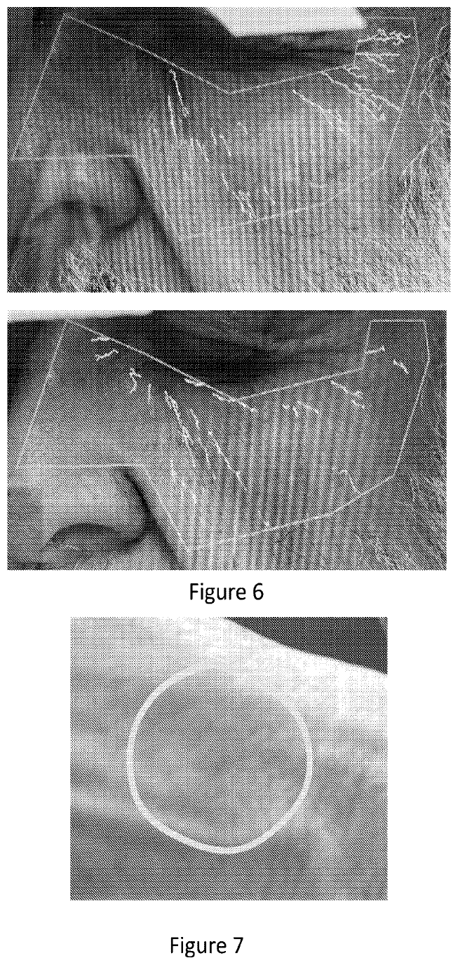

Administration of about 3 g of citrate daily for about 3 months was also found to reduce the appearance of a precancerous spot on the skin (FIG. 7).

1-72. (canceled)

73. A cosmetic method of preventing or reducing the effects of aging in a subject, the method comprising administering citrate to the subject; wherein preventing or reducing the effects of aging comprise preventing or reducing greying of hair, and wherein the citrate is administered to the subject by sublingual administration, intravenous administration, or oral administration.

74. A method of preventing or reducing the effects of aging in a subject, the method comprising administering citrate to the subject; wherein preventing or reducing the effects of aging comprise increasing muscle mass.

75. A cosmetic method of preventing or reducing the effects of aging in a subject, the method comprising administering citrate to the subject; wherein preventing or reducing the effects of aging comprise increasing muscle mass.

76. The method according to claim 74, wherein the citrate is administered to the subject by sublingual administration, intravenous administration, transdermal administration, or oral administration.

77. The method of claim 73, wherein the citrate is administered in combination with an antioxidant.

78. The method of claim 77, wherein the antioxidant is selected from melatonin, ethanol, a carotenoid, a flavonoid, ascorbic acid, vitamin E, and grape seed extract.

79. The method of claim 73, wherein the citrate is administered in combination with an anti-inflammatory agent.

80. The method of claim 79, wherein the anti-inflammatory agent is selected from non-steroidal anti-inflammatory drug (e.g. aspirin or ibuprofen), and a corticosteroid.

81. The method of claim 73, wherein the method comprises administering citrate to the subject more than once per day.

82. The method of claim 81, wherein the method comprises administering citrate to the subject at least three times per day.

83. The method of claim 73, wherein the citrate is administered as a salt, optionally wherein the salt comprises a cation selected from calcium, magnesium, potassium, and sodium.

84. The method of claim 73, wherein the method further comprises administering topical red or infrared radiation with a wavelength of between 600 and 900 nanometres to the subject.

85. The method according to claim 73, wherein the method comprises administering an anti-cholesterol agent to the subject.

86. The method according to claim 73, wherein the method comprises:
(i) monitoring inflammation levels in the subject; and
(ii) stopping or pausing citrate administration if inflammation levels exceed a threshold level.

87. The method according to claim 73 wherein the citrate is administered as a salt, wherein the method comprises:
(i) monitoring the level of at least one cation of the citrate salt in a biological sample obtained from the subject; and
(ii) stopping or pausing citrate administration if the level of said at least one cation of the citrate salt exceeds a threshold level;
optionally wherein the cation is selected from calcium, magnesium, potassium, and sodium.

88. The method according to claim 73, wherein the method comprises administering to the subject one or more supplements selected from vitamin D3, boron, zinc, quercetin, β-Nicotinamide mononucleotide (NMN), nicotinamide riboside (NR), sulforaphane, chia seeds, black turkey beans, psyllium husk, hyaluronic acid, berberine, ferulic acid, phosphorylated serine, 25 hydroxyvitamin G (25 (OH) D), aspirin, resveratrol, pterostilbene, pantethine, ketone ester, medium chain triglycerides (MCT), vitamin A, vitamin B, vitamin C, vitamin E or vitamin K (including vitamin K2 MK4, vitamin K2 MK7 and vitamin K2 MK9), fish oil, collagen, curcumin, carnosine, betaine, astaxanthin and grape seed extract.

89. The method according to claim 73, wherein the method comprises administering citrate to the subject if the level of C-reactive protein in a biological sample obtained from the subject exceeds a threshold level.

90. The method according to claim 73, wherein the citrate is administered systemically.

91. The method according to claim 77, wherein the anti-inflammatory agent and/or anti-oxidant is administered orally.

92. The method according to claim 73, wherein the citrate is formulated for time-release and/or sustained release.

93. The method according to claim 73, wherein the method comprises administering to the subject one or more histone deacetylase inhibitors, optionally wherein the one or more histone deacetylase inhibitors are selected from Berberine, Curcumin, Quercetin and Pterostilbene.

94. The method according to claim 73, wherein the method comprises administering to the subject a GSK3ß inhibitor, optionally wherein the GSK3ß inhibitor is lithium.

95. The method according to claim 73, wherein the method comprises administering to the subject acetate or an acetate precursor such as ethanol.

96. The method according to claim 73, wherein the method comprises administering to the subject a B vitamin, optionally wherein the B vitamin is selected from vitamin B1, vitamin B2, vitamin B3, vitamin B5, vitamin B6, vitamin B7, vitamin B9 and vitamin B12.

97. The method according to claim 73, wherein the method comprises administering to the subject copper.

98. The method according to claim 73, wherein the method comprises administering to the subject pantethine or vitamin B5.

99. The method according to claim 73, wherein the method comprises administering to the subject caffeine, optionally wherein the caffeine is administered topically.

100. The method according to claim 73, wherein the method comprises administering to the subject minoxidil.

101. The method according to claim 73, wherein the method comprises administering to the subject vanillin.

102. The method according to claim 73, wherein the method comprises administering to the subject Alpha Lipoic Acid.

103. The method according to claim 73, wherein the method comprises administering to the subject Rapamycin.

104. The method according to claim 73, wherein the method comprises administering to the subject an HMG reductase inhibitor.

105. The method according to claim 73, wherein the method comprises administering to the subject ribonucleotides.

106. The method according to claim 73, wherein the citrate is formulated for topical use, optionally wherein the topical formulation comprises a solvent, optionally wherein the solvent is DMSO.

107. The method according to claim 73, wherein the method comprises administering to the subject at least 0.5 g of citrate per day, optionally at least 2 g, at least 3 g, at least 4 g, at least 5 g, or at least 6 g of citrate per day.

108. The method according to claim 73, wherein the method comprises administering to the subject at least 10 ml of lemon juice per day.

109. A composition comprising citrate for use in a method according to claim 73.
